(19) United States
(12) Patent Application Publication
(10) Pub. No.: US 2006/0198814 A1
(43) Pub. Date: Sep. 7, 2006

(54) Anti-infectious hydrogel compositions

(75) Inventors: Rainer Gruening, Basking Ridge, NJ (US); Doug J. Perschbacher, Bloomsbury, NJ (US); Xin Qu, East Brunswick, NJ (US); David Buongiovanni, Manville, NJ (US)

Correspondence Address: HOFFMANN & BARON, LLP, 6900 JERICHO TURNPIKE, SYOSSET, NY, 11791, US

(73) Assignee: HYDROMER, INC.

(21) Appl. No.: 11/416,060

(22) Filed: May 2, 2006

Related U.S. Application Data
(62) Division of application No. 10/788,663, filed on Feb. 27, 2004.

(51) Int. Cl. A61K 31/77; A61K 31/715

(52) U.S. Cl. 424/78.3; 514/54

(57) ABSTRACT

The present invention provides a hydrogel composition capable of preventing the intrusion of micro-organisms into body cavities or body openings of mammals comprising of a poly(N-vinyl lactam), a polysaccharide and water.

### BACKGROUND OF THE INVENTION

Body cavities with openings to the periphery of a mammal, both natural cavities and those resulting from injury, have a high risk of microbial contamination. Infectious contamination could result in life-threatening consequences, particularly in immune compromised mammals. Microbial infections of, for example, the ear canal, the eye, the nail or hoof, the vagina, the teat, burns and lacerations are well known to physicians and veterinarians. Examples of organisms involved include gram-negative and gram-positive species, mycoplasma strains and a number of fungi. Frequent care and cleaning of body cavities and openings are required in order to minimize the risk of infections by these ubiquitous microbes.

An example of a body cavity that is prone to infections is the teats of dairy animals. Infection of the teats is termed mastitis. Although dairy mammals have a risk for mastitis throughout their milking cycle, dairy cows have a particularly high risk for mastitis during their dry periods. The dry period is approximately four to ten-weeks immediately preceding the delivery of a calf. This period is also known as the non-lactating period. Although during the dry period the cow is not at risk for contamination from milking machines, over fifty percent of teat infections occur during a cow's dry period. This high rate of infection occurs since a cow's immune response is diminished during the dry period. Additionally, the teat is distended during the dry period allowing microbes to penetrate the mammary gland more easily; and without the flushing lactation provides, the likelihood of infection increases. The residual milk protein in the teat provides a good feeding ground for microorganisms which cause mastitis.

Mastitis involves a wide range of environmental microorganisms including bacteria, fungi and a number of mycoplasma strains. The mastitis related bacteria recognized by the Food and Drug Administration (FDA) and the National Mastitis Counsel (NMC) include Staphylococcus aureus, Klebsiella spp., Streptococcus agalactiae, Pseudomonas spp., Streptococcus dysgalactiae, Corynebacterium bovis, Streptococcus uberis, Nocardia, Streptococcus bovis, Candida albicans, Escherichia coli, and Mycoplasma spp. Mycoplasma species include Mycoplasma bovis, Mycoplasma californicum, and Mycoplasma bovogenitalium. Additionally, Salmonella strains, Proteus vulgaris, Bordetella bronchiseptica, Pastorella multocida and others have received intensive research attention due to their frequent occurrences. The NMC in conjunction with the FDA, and several international health and safety agencies, have stated the importance of the control of the aforementioned microorganisms in the mastitis related dairy industry.

The consequences of mastitis during a cow's dry period include contamination of the newborn calf and of the subsequently produced milk, which leads to lower breeding results; lower milk production; and in severe cases, loss of the cow and calf. In the United States alone, mastitis costs the dairy industry close to $3 billion a year, or about $300 per cow. The costs include drugs, veterinary treatments, and discarded milk or decreased milk production.

A number of methods for mastitis prevention have been suggested in research publications and patent literature including general hygiene programs, sanitizer products, milking cycle barrier dips, long lasting dry cow dips, antimicrobial barrier products, systemic and locally applied antibiotics, internal teat treatments and antibiotic teat canal plug systems. However, current methods for controlling mastitis have many shortcomings.

For example, antibiotics may contaminate both the milk and meat of a cow. Also, antibiotics do not provide a complete prevention of infection. Furthermore, extensive use of antibiotics leads to resistance by microorganisms, thereby compelling the development of new antibiotics.

Also, most of the currently-used teat dips are used during a mammals lactating period. For example, effective teat dip compositions used during the regular milking cycle of a dairy cow are described in U.S. Pat. Nos. 6,395,289 and 6,203,812 (Hydromer, Inc. Branchburg, N.J.). These compositions are hydrophilic polymeric blends, which provide effective and long-lasting barrier properties while allowing for rapid removal of the composition prior to milking. The exterior of a mammalian teat is dipped into the composition. However, the physical consistency and properties of such teat dips make them unsuitable for teat canal treatment. For example, since these dips do not gel over readily, they would tend to run out of the canal.

Other teat dip compositions used during a mammal's lactating period are disclosed in U.S. Pat. Nos. 4,113,854 and 5,017,369. Applied externally, these compositions form thick films which seal-off the end of a teat canal. These compositions include latex. As a result of the latex, these compositions remain viscous and sticky thereby not allowing for teat canal treatment. Also, latex may be toxic. In addition to the contamination of milk, latex can elicit allergic reactions in humans.

Despite the fact that over fifty percent of mastitis cases occur during a cow's dry period, only a few products are on the market which are specifically designed for dry cow teat protection. Treating a dairy animal during its dry period would complement the treatment during the milking cycle

Dry cow products currently on the market have several shortcomings. For example, most of these products do not address treatment of the teat canal. Treatment of the teat canal is important because residual milk protein in the canal serves as an excellent breeding ground for microorganisms. Another shortcoming of some of the currently available dry cow teat canal treatments is that they require complex process steps, such as irradiation, heat, catalysts or other specific additives to form a useful shape-maintaining plug substance. Further shortcomings are that they are not stable at a wide range of temperatures and/or at changing moisture conditions.

U.S. Pat. Nos. 6,254,881, 6,340,469 and 6,506,400 disclose an antibiotic-free formulation for the prophylactic treatment of mastitis in dry cows. The formulation is infused into the teat end to seal the teat canal against mastitis-causing microorganisms. The formulation consists of approximately 65% by weight of bismuth sub-nitrate in a gel based on aluminum stearate. Although these patents claim an antibiotic-free formulation, the use of antibiotics in conjunction with the formulation is recommended by the NTMC. Among the disadvantages of this formulation is that the bismuth sub-nitrate thickens in cold weather thereby hindering its ability to be sufficiently infused into the teat canal. Additionally, since these formulations may interfere with the mechanics of milking machines, these formulations are required to be stripped out manually from the teat canal prior to machine milking.

U.S. Patent Application 2003/0060414 describes a method of preventing contamination of a teat during administration of a sealant by introducing a sterilizing agent into a teat before delivering a sealant. The sterilizing agent is a water miscible gel, oil-based gel or oil based paste containing a bacteriocin, e.g., Lacticin 3147. The sterilizing agent may include thickeners and/or other excipients. The consistencies of these sterilizing agents are paste-like, and change shape irreversibly upon certain forces. Thickeners are used in these agents, in part, to preserve the shape of these pastes.

U.S. Pat. No. 4,472,374 describes veterinary compositions for reducing mammary infections during the dry period. These compositions contain a siloxane elastomer with an incorporated antibacterial agent. These compositions are of sufficiently low viscosity to facilitate application to the streak canal; and these compositions remain in place during the dry period and can be milked-out at the onset of lactation. However, complex processes are necessary to make these compositions, including the use of curing catalysts. Such catalysts pose toxicological concerns since these catalysts can leach out as highly reactive compounds.

Long-lasting film forming hydrophilic polymer blends are described for dry cow mastitis therapy in U.S. Pat. No. 6,440,442 (Hydromer, Inc. Branchburg, N.J.). The films form on the outside of the teat and functions as a barrier to prevent infection. The main components of these blends are polyurethane and poly(N-vinyl lactam). Since these dips are viscous, they are not readily suitable for infusion into the interior teat canals.

Despite many decades of intensive research on the prevention of mastitis and the availability of numerous teat dip products, sanitizers and antibiotics, dry cow mastitis infections still have a significant negative impact on the economics of milk production. There is an increasing need for effective dry cow treatments to complement mastitis control treatments used during the lactating periods. Such dry treatments would improve economics and food hygiene of the milk production, and minimize the use of antibiotics.

### SUMMARY OF THE INVENTION

The present invention is directed to novel hydrogel compositions capable of preventing the intrusion of microorganisms into body cavities or body openings of a mammal. The compositions have a specific ratio of a polyvinyl lactam to a polysaccharide which forms a gelatinous composition with water. The compositions optionally comprise consistency-modifying agents, performance-modifying agents, cross-linkers, and therapeutic enhancing agents.

The hydrogel compositions are suitable for being transferred into natural body cavities of mammals, such as the teat canal of a dairy cow; and accidental skin cavities caused by injury such as cuts, burns and disease. The compositions are applied to body cavities or openings by means of infusion tools, preferably a plastic syringe. The hydrogel compositions form a barrier or a sealant for the prevention of intrusion of infection-causing microorganisms. For example, the hydrogel compositions prevent the contamination of a teat canal of a dry cow from infections by environmental mastitis related microorganisms. Simultaneously, the hydrogel compositions also sanitize, disinfect, prevent inflammation, and promote healing of the interior walls of a body cavity or body opening. Such sanitizing/disinfecting activity occurs without the inclusion of antimicrobial/antibiotics.

The hydrogel compositions of the present invention provide several advantages over currently used teat dip treatments

For example, most teat dip treatments are formulated for use during a cow's lactating period; whereas, over fifty percent of all mastitis cases are detected in the dry period of dairy cows. The hydrogel compositions of the present invention are formulated for use during a cow's dry period. Additionally, most currently available dry cow treatments require the use of antibiotics. The hydrogel compositions of the present invention provide disinfecting/sanitizing activity without the need of antibiotics. Minimizing the use of antibiotics lowers the risk of antibiotic side effects, avoids long waiting periods after antibiotic applications and decreases the risk of developing antibiotic resistance in microorganisms.

Moreover, unlike most currently available dry cow treatments which require complex processing steps, such as curing, and catalytic reactions, the hydrogel compositions of the present invention are made by a simple mixing procedure. Furthermore, unlike most currently available dry cow treatments, the hydrogel compositions of the present invention are stable in a wide temperature range.

### DETAILED DESCRIPTION OF THE INVENTION

The present invention relates to biocompatible lubricious, hydrogel compositions which are suitable to fill body cavities and body openings of mammals. The hydrogel compositions are in the form of reversible or irreversible hydrogels. The hydrogel compositions function as body cavity or body opening sealants, and/or sanitizers.

Throughout this specification, there are ranges defined by upper and lower boundaries. Each lower boundary can be combined with each upper boundary to define a range. The lower and upper boundaries should each be taken as a separate element.

The hydrogel compositions of the present invention comprise a poly(N-vinyl lactam); a polysaccharide, and water. Preferably, the range of the ratio of the amount by weight of the poly(N-vinyl) lactam to the amount by weight of the polysaccharide has an upper boundary of approximately 75:1. Examples of other upper boundaries include about 1; 50:1; 30:1; 20:1; 15:1; 13:1; 12:1; and 1:2.

Preferably, the range of the ratio of the amount by weight of the poly(N-vinyl) lactam to the amount by weight of the polysaccharide has a lower boundary of approximately 1:10. Examples of other lower boundaries include about 1:5; 1:3, 1:1; 5:1; 12:1; 13:1; 15:1; 20:1; 30:1; and 50:1.

The poly(N-vinyl lactam) of the hydrogel compositions of the present invention can be any type of poly(N-vinyl lactam), such as, for example, a homopolymer, a copolymer, or a terpolymer of N-vinyl lactam, or mixtures thereof. Examples of poly(N-vinyl lactam) polymers suitable for use in the hydrogel compositions include N-vinylpyrrolidone, N-vinylbutyrolactam, N-vinylcaprolactam, and mixtures thereof. An example of a preferred poly(N-vinyl lactam) homopolymer is polyvinylpyrrolidone (PVP).

Examples of poly(N-vinyl lactam) copolymers and terpolymers include N-vinyl lactam polymers which are copolymerized with vinyl monomers. Examples of vinyl monomers include acrylates, hydroxyalkylacrylates, methacrylate, acrylic acids, methacrylic acids, acrylamides, and mixtures thereof. The copolymerization of the N-vinyl lactams with vinyl monomers allows for modification of the consistency of the hydrogel compositions.

Examples of preferred poly(N-vinyl lactam) copolymers include vinylpyrrolidone copolymer and an acrylamide copolymer. Examples of preferred terpolymers include vinylpyrrolidone terpolymers, vinylcaprolactam terpolymers, and dimethylaminoethyl methacrylate terpolymers.

Preferably, the poly(N-vinyl lactams) used in the hydrogel compositions of the present invention are commercially available poly(N-vinyl lactams), and do not require any pretreatment before use in the hydrogels. For example, preferably, the poly(N-vinyl lactams) are not treated to induce the openings of their lactam rings.

In one embodiment, the hydrogel compositions of the present invention do not contain a polymer of an acid, e.g., polyacrylic acid, or an acid forming compound such as an anhydride.

The polysaccharide used in the hydrogel compositions can be any polysaccharide. For the purposes of this specification, a polysaccharide includes any polysaccharide and any polysaccharide derivative. Examples of polysaccharide suitable for use in the composition include chitin; deacetylated chitin; chitosan; chitosan salts; chitosan sorbate; chitosan propionate; chitosan lactate; chitosan salicylate; chitosan pyrrolidone carboxylate; chitosan itaconate; chitosan niacinate; chitosan formate; chitosan acetate; chitosan gallate; chitosan glutamate; chitosan maleate; chitosan aspartate; chitosan glycolate; quaternary amine substituted chitosan salts; N-carboxymethyl chitosan; O-carboxymethyl chitosan; N,—O-carboxymethyl chitosan; equivalent butyl chitosan derivatives; cellulosics, alkylcellulose; nitrocellulose; hydroxypropylcellulose; starch; starch derivatives; methyl gluceth derivatives; collagen, alginate; hialuronic acid; heparin; heparin derivatives; and combinations thereof.

The combined poly(N-vinyl lactam) and polysaccharide of the invention is hydrophilic, and is capable of absorbing many times its weight in water. The water content of the composition can vary depending on the particular use of the composition, as would be known by a skilled artisan. Preferably, the range of the water content in the composition has an upper boundary of about 90 wt % water. Examples of other upper boundaries include about 75 wt % water and 65 wt % water. Preferably, the range of the water content in the composition has a lower boundary of about 25 wt %. Examples of other lower boundaries include about 45 wt % water and 55 wt %. As the water content of the hydrogel compositions increase, the hydrogel compositions become softer.

In some embodiments of the invention, some of the water of the composition is replaced by an alcohol. Approximately 15 wt % to 75 wt %, 35 wt % to 65 wt %, or 45 wt % to 55 wt % of the water can be replaced with alcohol. Preferred examples of alcohols include ethyl alcohol and isopropyl alcohol.

The hydrogel compositions comprising the combination of poly(N-vinyl lactam) and polysaccharide unexpectedly have a consistency which enable the hydrogel compositions to efficiently fill, and to remain in, body cavities/openings. For example, in dairy animals, the hydrogels stay in the teat canals for extended periods of time even while the animals move about or bed down. Additionally, the consistency of these hydrogels allows for them to be squeezed out in total when needed or desired.

After the compositions of the present invention form a gel, they can be broken up and then, surprisingly, form a gel again in a few hours. Thus, these hydrogels are fully reversible. While not being limited by a theory, it is believed that the hydrogen bonds in these hydrogels are temporarily broken when such hydrogels are forced through small holes of applicators. The hydrogen bonds fuse together again after a few hours.

In some embodiments of the invention, the hydrogel compositions can further comprise at least one consistency modifying agent, a performance modifying agent, a cross-linker, or mixtures thereof.

Up to approximately 5 wt %, 10 wt %, 20 wt %, 30 wt %, 40 wt %, 50 wt %, 60 wt %, 70 wt %, 80 wt %, or 90 wt % of the poly(N-vinyl lactam) can be replaced with the consistency and/or performance modifying agents. For example, in a formulation comprising polyvinyl pyrrolidone (PVP) and chitosan, or chitosan derivatives, preferably about 50 wt % of the PVP is replaced with consistency and/or performance modifying agents.

Examples of preferred consistency modifying and/or performance modifying agents include polyvinyl alcohol; polyvinyl acetate; pblyethylenoxide, poly(2-hydroxyethyl methacrylate); methyl vinyl ether-co-maleic anhydride; poly(ethylene-co-vinyl acetate); polyethylene glycol diacrylate; poly(N-isopropyl acrylamide); polyurethane; dimethicone; polyglycol ester copolymers, adhesive prepolymers, polyethylenimine; polypeptides; keratins; copolymers of polyvinylpyrrolidone/polyethyleneimine; polyvinylpyrrolidone/polycarbamyl/-polyglycol ester (Aquamer® H-1212, H-1511, H-2012, A-1212); polyvinylpyrrolidone/dimethylaminoethylmethacrylate/polycarbamyl/polyglycol ester (Aquamere® C-1011, C-1031);

polyvinylpyrrolidone/dimethiconylacrylate/polycarbamyl/-polyglycol ester (Aquamere® S2011, S-2012); (PECOGEL equivalents of the Aquamere® products); lecithin; and copolymers, derivatives and combinations thereof. U.S. Pat. Nos. 4,642,267; 4,769,013; 5,837,266; 5,851,540; and 5,888,520 assigned to Hydromer, Inc., are incorporated by reference in their entireties. For example, U.S. Pat. Nos. 4,642,267 and 4,769,013 describe lubricity/hydrophilicity copolymers with performance modifying therapeutic agents and polymers; and lubricious, hydrophilic, antimicrobial coatings for the tip of a gel syringe application device. U.S. Pat. Nos. 5,837,266; 5,851,540, and 5,888,520 describe dermatological acceptable polymers and copolymers with therapeutic agents and barrier performance against dermatitis:

The copolymers of polyvinylpyrrolidone/polyethyleneimine, polyvinylpyrrolidone/polycarbamyl/polyglycol ester (Aquamere® H-1212, H-1511, H-2012, A-1212), polyvinylpyrrolidone/dimethylaminoethylmethacrylate/polycarbamyl/polyglycol ester (Aquamere® C-1001, C-1031), polyvinylpyrrolidone/dimethiconylacrylate/polycarbamyl/-polyglycol ester (Aquamere® S2011, S-2012) and their PECOGEL equivalents are well known as cosmetic intermediates. (Phoenix Chemicals, N.J.) The Aquamere® copolymers are known to have unique hydrophobizing properties. In particular, these copolymers provide unique polymeric encapsulating effects which slow down the release of actives ingredients such as UV absorbers, dyes, colorants, oxidizers, preservatives, antimicrobials, antibiotics and drugs. For example, the dimethiconylacrylate version of the Aquamere® S2011, S-2012 copolymers are known to form inclusion complex polymers, which can retard the solubility of emulsified actives.

The Aquamere® copolymers are hydrophobic viscous liquids and have been thought to be unsuitable for the use as gels for infusion into body cavities or body openings. However, it has now been surprisingly discovered that gelation of the hydrogel compositions of the present invention is still achieved while replacing up to 90 wt % of the water in the compositions with the Aquamere® copolymers. The Aquamere® copolymers function to slow the release of additives, e.g., of therapeutic agents and antimicrobials. The addition of Aquamere® copolymers to the hydrogel composition affects the amount of poly(N-vinyl lactam) used in the composition. For example, if an original formulation is 35 wt % PVP, 2 wt % chitosan, and 63 wt % water, then a corresponding Aquamere® formulation is 25 wt % PVP, 10 wt % Aquamere® copolymers, 2 wt % chitosan, and 63 wt % water. Lecithin, well known in the food and cosmetic industry, has functions similar to the Aquamere® copolymers.

For additional performance enhancement, the hydrogel compositions can optionally contain humectants, e.g. glycerin.

The hydrogel compositions of the present invention can be either a reversible or irreversible hydrogel. The components of a reversible hydrogel dissolve in water. The components of an irreversible hydrogel gel do not dissolve in water due to the presence of cross-linking agerits (i.e. cross-linkers) which provide, depending on the amount used, a certain amount of irreversible links.

Cross-linkers enhance the ability of the hydrogel compositions to maintain their original shape, remain in a body cavity or opening, and/or enhance the ability of the hydrogel compositions to be easily removed from the cavity or opening. For example, cross-linkers enhance the ability for the hydrogel compositions to remain in the teat canal, and enable the easy removal from the teat by squeezing. Examples of cross-linkers which are suitable for use in the composition include glutaraldehyde, genipin, aziridine derivatives, carbodimid derivatives, colloidal silica, colloidal alumina, colloidal titanium dioxide, polyaminosilanes, epoxies, primary polyamines, dialdehydes, polyaldehydes from acrolein reaction products, paraformaldehyde, acrylamides, polyethylenimines, and combinations thereof.

Cross-linkers can be used in any amount which provides the hydrogel compositions with desired consistencies. For example, the composition can comprise up to about 2 wt %, 3 wt %, 4 wt %, 5 wt %, or 8wt % of a cross-linker.

The hydrogel compositions comprising poly(N-vinyl lactam) and polysaccharide surprisingly have sealant and sanitizing/disinfecting properties. In some embodiments of the invention,-the hydrogel compositions can further comprise at least one therapeutic performance enhancing agent. Therapeutic performance enhancing agent can comprise up to about 3 wt %, 7 wt %, 10 wt %, 15 wt %, or 20 wt % of the composition.

Examples of therapeutic performance enhancing agents which are suitable for use in the composition include antimicrobials; antibacterials; antifungals; anti-candidiasis agents; growth stimulating agents; disinfecting agents; biocides; bactericides; preservatives; virucides; spermicides; germicides; sterilants; sanitizing ingredients; deodorizers; antiseptics; sporicides; pharmaceuticals; veterinary preparations; antibiotics; anti-inflammatory agents; natural ingredients; humectants; cosmetic ingredients; soothing agents; vitamins; and combinations thereof.

Some specific examples therapeutic performance enhancing agent include antimicrobial silver salts, silver zeolites, silver sulfadiazine, ethyl alcohol, isopropyl alcohol, benzyl alcohol, propionic acid, sorbic acid, salicylic acid, undecanoic acid, bleaches, iodine, iodophor, potassium iodide, dodecyl benzene sulfonic acid, peroxides, bronopol, terbinafine, miconacole, econacole, clotrimazole, tolnaphthate, triclosan, trichlocarban, quaternary ammonium compounds, benzalkonium halogenides, polyquats; polyquaternium derivatives (e.g., polyquaternium-28); formaldehyde releasing compounds, hexetidin, chlorhexidine, chlorhexidine derivatives, zinc pyrithione, zinc oxide, zinc propionate, parabens, phenoxyethanol, octoxynol-9, nonoxynol-9, ricinoleic acid, phenol mercuric acetates, sulfur, lactic acid, acyclovir, idoxyumidine, ribavirin, vidarabine, rimantadine, aspirin, vitamin A and vitamin A derivatives, vitamin E and vitamin E derivatives, vitamin C and vitamin C derivatives, betacarotin, betamethasone, dexamethasone, cortinone, glycerin, and combinations thereof.

The therapeutic performance-enhancing agents from the group of natural ingredients include, for example, plant or seed extracts, plant extract derivatives or herbal preparations or combinations thereof. Examples of natural ingredients include extracts of rosemary, echinechea, nettle, fennel, juniper, ginseng borage, gelsemium, hamamelis, poke root, arnica, aconite, apis, baptisia, thuja and aloe (barbadensis, vera, capensis), green tea, nasturtium, bryonia, eupatorium, and chamormile. Further examples include essential oils of red thyme, allspice, cinnamon and savory.

Examples of antimicrobial silver salts include silver iodide, composites of silver chloride upon titanium (IV) oxide, silver lactate, silver citrate, silver zeolites, silver sodium hydrogen zirconium phosphate and silver sulfadiazine.

Preferably, in order to minimize the build-up of resistance to the ingredients, combinations of different antimicrobials, antibiotics, and anti-inflammatory agents can be used in the hydrogel compositions. Also, natural plant and seed extracts can be used in combination with the anti-inflammatory agents, antimicrobials, and antibiotics to further minimize the build-up of resistance.

The amount of the therapeutic performance enhancing agents in the hydrogel compositions is within the effective range of the individual agents. For example, the hydrogel compositions with an effective concentration of a spermicide are suitable for use as contraceptive hydrogels. Typically, the hydrogel compositions of the invention comprise up to about 3 wt %, 7 wt %, 10 wt %, 15 wt %, or 20 % of therapeutic performance enhancing agents.

In some embodiments of the invention, the hydrogel compositions can further comprise a dye, such as, for example, a control dye, a food dye, a cosmetic dye, a FD&C dye or a D&C approved dye.

In some embodiments of the invention, the hydrogel compositions can further comprise a radio-opaque additive, such as, for example, barium sulfate, iodine organics, iodine polymers, iodine contrast media, bismuth organics, tungsten particles and mixtures thereof.

In another aspect, the present invention provides a method of inhibiting or preventing the intrusion of microorganisms into a mammalian body cavity or opening; and/or reducing or eliminating the level of microorganisms in such cavity or opening. The method comprises applying the hydrogel compositions of the present invention into a body cavity or opening.

A body cavity or opening can be naturally-occurring. Examples of natural-occurring body cavities or openings include an ear canal, eye, nasal canal, mouth, dental openings, genital opening, rectal opening, wrinkle or gland opening. An example of a gland opening is the teat canal of the milk gland of a dairy animal. The teat canal is also called the streak canal or milk canal.

A non-naturally-occurring body cavity or opening can be a result of a laceration, a burn or a disease. Examples of such cavities or openings include puncture wounds, stabbing wounds, scabs, diabetic ulcers, periodontal lesions, herpes sores, cold sores, blisters, superficial to severe burns, etc.

The composition can be used with any mammal, including, for example, humans, zoo animals, pets, and farm animals. An example of a farm animal for which the composition is particularly useful is dairy cows.

Once applied to a body cavity or opening, the hydrogel compositions preferably have a dual function, i.e. as a sealant and as a sanitizer.

In particular, the hydrogel compositions function as sealants by preventing/inhibiting intrusion of microorganisms into a cavity or opening. The hydrogel composition forms a hydrophilic tissue-friendly barrier which provides long-lasting service. The compositions exhibit specific tackiness enabling the hydrogel compositions to stay in place for extended periods of time.

Also, the hydrogel compositions, due to their unique formulation, function as sanitizers/disinfectants by reducing or eliminating the level of microorganisms in a cavity or opening. Surprisingly, the hydrogel compositions are capable of reducing or eliminating the level of microorganisms without the use any therapeutic enhancing agents, such as antibiotics and antimicrobials.

The hydrogel compositions can be applied in any manner which would enable the hydrogel compositions to efficiently fill, and to remain in, body cavities/openings. For example, the composition can be applied with a spatula; by hand; by an injection device; by infusion devices, such as plastic syringes; by plungers; or by applicators. Preferably the hydrogel compositions are applied with plastic syringes. Preferably, the syringes have suitable tubular openings adjusted to the size of the intended area of application. The hydrogel compositions can be applied once, and replaced if desired or necessary.

In some methods of application, such as by injection, the hydrogels break apart during application into cavities. Once applied, it has been surprisingly found that the hydrogels fuse together again when in place. Without being limited to a theory, it is believed that the hydrogen bonds of the hydrogels are temporarily broken when the hydrogels are forced through small applicator holes. After few hours, surprisingly, the bonds fuse together again.

The hydrogel compositions of the present invention are particularly useful as teat canal sealants for dairy mammals. In particular, the hydrogel compositions are useful as teat canal plugs for cows during their dry period. The dry period runs approximately from about four to ten weeks immediately preceding the delivery of a calf. The hydrogel compositions function as a sealant by temporarily plugging the teat, thereby preventing intrusion of mastitis-causing microorganisms. The hydrogel compositions also function as sanitizers/disinfectants of the teat canal by reducing/eliminating mastitis-causing microorganisms within the teat canal.

The hydrogel compositions are preferably applied into the teat canal, i.e. streak canal, by an infusion device. Any infusion device suitable for intramammary administration can be used, or readily adapted for such use. An example of a suitable infusion device is a syringe known as a “mastitis applicator.” Syringes can have either a plastic cannula or a wide-bore needle.

In some embodiments, a therapeutic enhancing agent can be injected separately from the hydrogel composition. For example, in the case where the hydrogel composition is used in conjunction with an antimicrobial, the composition and the antimicrobial can be infused simultaneously using a normal one cylinder syringe, fitted with a suitable tip, such that the antimicrobial solution enters the body cavity, e.g., teat, first, followed by the composition. Alternatively, the antimicrobial solution and the hydrogel composition can be infused using separate syringes.

The hydrogel composition can be placed into a teat canal by infusing about one cm³ into each teat canal. Once the hydrogel compositions are placed into a teat canal and they have gelled, the hydrogels maintain their shape. Due to certain tackiness, the hydrogel plugs can stay in the teat canal for extended periods of time even when the cows move about or bed down. For example, the hydrogels preferably remain in the teat canal during the dry period for about one to ten days. The hydrogels can be squeezed out in total if needed or desired.

Dry cow treatment procedure by intramammary infusion is a potentially dangerous procedure. The danger lies in unsanitary infusion practices, which can introduce additional environmental organisms into the udder posing increased risk of mastitis infection. It is therefore recommended to sterilize the hydrogel compositions and the infusion devices.

For example, for prevention of cross-contamination during application of the hydrogel compositions, the tips of the infusion devices are coated with lubricious, antimicrobial coatings known in the art. For example, antimicrobial lubricious coatings for medical devices are disclosed in U.S. Pat. Nos. 4,642,267; and 4,769,013. Preferably, the antirmicrobials are silver based compositions. Preferably, the inside of the tip of the device is also coated with the lubricious coating for improving the ease with which the hydrogel compositions are forced through the opening of the infusion devices.

Once applied, the hydrogel compositions form a barrier or a sealant for the prevention and/or inhibition of intrusion of microorganisms into the teat. The hydrogel compositions prevent the contamination of a teat canal of a dry cow from infections by environmental mastitis related microorganisms. Simultaneously, the composition plug sanitizes, disinfects and prevents inflammation of the interior walls of a body cavity or body opening. Sanitizing and disinfecting occur without the optional addition of antibiotics/antimicrobials.

The hydrogel compositions can be used in conjunction with a hydrophilic external film forming product for additionally protection of the teat. For example, the hydrogel compositions can be infused into the teat canal, while simultaneously applying a dry cow teat dip, such as the dry teat dip described in U.S. Pat. No. 6,440,442, to the outside of the teat.

In one embodiment, the invention provides a contraceptive hydrogel comprising a poly(N-vinyl lactam), a polysaccharide, water and a spermicide, wherein the ratio of the amount by weight of the poly(N-vinyl) lactam to the amount by weight of the polysaccharide is about 75:1 to 1:5; about 50:1 to 1:1; or about 30:1 to 5:1, and wherein the composition comprises about 25 wt % to 55 wt % water. In this embodiment, the hydrogel comprises an effective concentration of a spermicide to function as a suitable contraceptive.

The hydrogel compositions of the present invention can be produced by a variety of methods. Preferably, the poly(N-vinyl lactam) component and the polysaccharide component of the hydrogel compositions are preformulated in separate solutions. In a preferred embodiment, the two solutions are approximately equal in volume. The solutions can be aqueous solutions or aqueous/alcohol solutions.

Any optionally added ingredients, i.e. consistency and/or performance-modifying copolymers, cross-linkers, therapeutic performance enhancing agents, dyes and/or radio-opaque additives, are preferably added in equal amounts to the poly(N-vinyl lactam) preformulated solution and the polysaccharide preformulated solution prior to combining the two solutions. Alternatively, all the optionally added ingredients can be put into either the poly(N-vinyl lactam) preformulated solution or the polysaccharide preformulated solution prior to combining the two solutions. Also, any fraction of the optionally added ingredients can be put into either preformulated solution prior to combining the two solutions. For example, twice as much cross-linker can be put into the poly(N-vinyl lactam) preformulated solution than the polysaccharide preformulated prior to combining the two parts needed for the total composition.

The preformulated solutions are mixed in any manner which allows homogeneous mixing of the two solutions prior to the time when gelling starts to occur. For example, mixing can be accomplished using a screw mixer or simply mixing the two parts in a vessel.

The initial gelling of the composition can occur from a few seconds to a few minutes after mixing of the two solutions. No other process steps, curing, or additional additives are needed in order for gelation to occur. For example, irradiation, heat, or catalysts are not required for formation of these hydrogels.

The hydrogel compositions are preferably allowed to completely gel at ambient temperature for about two to ten hours. The composition can then be placed in suitable devices for convenient applications into body cavities or body openings of mammals.

The hydrogel compositions of the present invention have various advantages over general competitive hydrogel types. The hydrogel compositions, exhibiting the desired performance and consistency, form simply by physically mixing the major components in the specified ratios. The addition of performance enhancing agents and/or consistency modifying agents is generally not necessary. The hydrogel compositions are formed over a period of time ranging from a few second to a few minutes. No other process steps, or other additives, are needed. They can be molded into shapes to fit an existing product design. They can be used as hydrophilic plug-forming consistencies with unique protective barrier properties. The gels have moisturizing and absorbent properties and are compatible with a broad range of cosmetic and drug ingredients. They absorb water, saline, derma- or other body fluids, provide cooling and soothing moisture barriers and enhance healing of damaged skin. Alone or with a variety of antimicrobials or antibiotic agents, anti-inflammatory, anti-candidiasis agents or related pharmaceutical or veterinarian preparations they contribute to the sanitization of mammalian body cavities/openings, and simultaneously prevent subsequent intrusion of microbes, such as bacteria, fungi, spores, germs, viruses and the like.

The hydrogel compositions of the present invention have shown good inertness within a relatively wide pH range around neutral pH. They are stable for at least one year in the appropriate evaporation-proof package. The results of long-term tests with the active ingredients formulated in these hydrogel compositions do not show any interaction or incompatibility with vitamins and their derivatives, plant or seed extracts, phospholipids, astringents, antimicrobials, antibiotics, anticandidiasis agents or other drug related pharmaceuticals, transdermal ingredients, skin-whiteners, green tea, anti-wrinkle actives, alpha hydroxy acids or cooling agents.

### EXAMPLES

Microbial Testing

The hydrogels of the present invention were tested for their antimicrobial/biostatic potential by a laboratory test method, which provides a qualitative and semi-quantitative procedure for the evaluation of antimicrobial activity by diffusion of the antimicrobial agent through agar. The method is derived from the “Parallel Streak Method” which is based on the Antibacterial Activity Assessment of Textile Materials; AATCC Test Method 147-1998.

The cultures were prepared fresh overnight. The test organisms used were Escherichia coli, ATCC# 25922 and Staphylococcus aureus, ATCC# 29213. The organisms were incubated with Tryptone Soy Broth (TSB) at 37° C. the day before the test. The bacterial cell suspension in TSB was >10⁷ cells per ml. On the day of test, hot agar samples were cooled in sterile tubes and then 0.1 ml of the individual culture was added to the melted agar. The agar samples were poured onto plates after mixing, allowed to gel and then test samples of hydrogels of the present inventions were placed on top of the agar. Incubation was then continued for one and 5 days and the zone of inhibition of bacterial growth approximated around each sample.

All percentages in the examples are weight percentages unless otherwise specified.

### Example 1

Method of Making a Hydrogel

1.4 grams propylene glycol and 3.0 grams of a 20% aqueous solution of a block copolymer of ethylene oxide and propylene oxide (Pluronic F88, BASF Corporation) were added to 8.6 grams of a 25% water solution of polyvinylpyrrolidone (PVP) (Kollidon K90, BASF Corporation). To that solution, 5 grams of a 3% aqueous solution of chitosan neutralized with pyrrolidone carboxylic acid (Kytamer PCA, Amerchol Corporation) were added. The mixture was stirred for a few minutes and transferred into plastic syringes for cavity applications.

### Example 2

Method of Making a Hydrogel

5.0 grams of a 20% solution of PVP in water were mixed with 5.0 grams of a 2% solution of N,O-carboxymethyl chitosan (NOCC, Nova Chem Ltd.). The mixture was poured into a hemispherical mold. It set in 10 seconds at room temperature to form a mildly tacky, non-flowable gel. The gel was pliable and relatively non-adherent to a wound.

### Example 3

Method of Making a Hydrogel

A solution of 5.0 g of 20% PVP, 5 grams of deionized water, 5.0 g of 2% neutralized chitosan, 0.25 grams of polyethylene glycol (carbowax 400, Union Carbide Corporation) as a plasticizer and 0.25 grams of a block copolymer of ethylene glycol and propylene glycol (Pluronic F88, BASF Corporation) were gently mixed until gelation occurs.

### Example 4

Variations in the Concentration of the PVP Portion of the Hydrogel

A solution of 20 g of a 30% PVP and 5% polyvinylpyrrolidone/dimethiconylacrylate/polycarbamyl/polyglycol ester in deionized water was mixed with 20 g of a 2.0% chitosan solution in deionized water. In a few minutes a hydrogel of firm tacky consistency was formed.

The formulation of the 20 g PVP solution was changed to 25% PVP and 10% polyvinylpyrrolidone/dimethiconylacrylate/-polycarbamyl/polyglycol ester. Again a firm hydrogel was obtained after a few minutes.

The formulation of the 20 g PVP solution was changed to 17.5% PVP and 17.5% polyvinylpyrrolidone/-dimethiconylacrylate/-polycarbamyl/polyglycol ester. After a few minutes, a firm tacky gel is formed.

A complete replacement of PVP with polyvinylpyrrolidone/dimethiconylacrylate/-polycarbamyl/polyglycol ester does not form a gel with the 2% solution of chitosan.

### Example 5

Antimicrobial Activity

A solution of 10 g of a 35% PVP in deionized water was mixed with 10 g of a 2.0% chitosan solution in deionized water where both solutions contained 1% of an antimicrobial silver composition available on the market under the name AlphaSan by Milliken.

The composition gels shortly after the two parts are combined in a 1 to 1 ratio. The gel is transferred into a 5 cc graduated plastic syringe. For antimicrobial efficacy testing, the gel was transferred into the standard test container in an agar petri dish. After 1 day and after 5 days, no growth of either Escherichia coli or Staphylococcus aureus was observed. A zone of inhibition of about 2 mm was formed by S. aureus. No actual zone of inhibition was detected for E. coli.

### Example 6

Method of Making a Hydrogel

44 g of a solution of 35% PVP and 6 g of a 40% aqueous polyurethane solution were mixed with 0.25% chitosan to yield 50.25 grams of a hydrogel composition which gels in a few minutes to a consistency with a tack which makes it suitable for infusion into body cavities, body openings, e.g., glands.

### Example 7

Antimicrobial Activity

20 g of an aqueous 35% PVP solution containing 0.1% Triclosan was mixed with 20 g of a 2% aqueous chitosan solution also containing 0.1% Triclosan. The gel was transferred into a plastic syringe and applied in form of a standard lump of 1×1×0.5 cm³ for antimicrobial testing. After 1 day and 5 days, no growth of either E. coli or S. aureus was observed. A zone of inhibition of about 6 to 9 mm for E. coli and 9 to 10 mm for S. aureus was observed.

### Example 8

Effect of Cross-Linker

To 44 g of a solution of 35% PVP and 6 g of a 40% aqueous polyurethane solution, as in Example 6, 0.2% commercially available genipin was added. 0.25% chitosan was prepared according to Example 6. Prior to mixing, both parts were stained with a few drops of a 0.1 Crystal Violet solution for better optical visibility. For testing the adhesion of the cross-linked and non-cross-linked gel in a stimulated teat canal, the finished hydrogels of this Example and from Example 6 were infused into a 20 cm long medical tube of about 3 mm D. The amount of about 3 cm gel in length on each end of the tube was injected. Clamped at the center of the tube and rotated with increasing rpm up to 600 rpm, the non-cross-linked hydrogel of Example 6 was able to stay in place up to 600 rpm; whereas the genipin cross-linked hydrogel was thrown out at about 450 to 500 rpm.

A hydrogel of Example 5 stayed in place for up to about 700 to 800 rpm

### Example 9

Antimicrobial Activity

20 g of an aqueous 35% PVP solution containing 1% aspirin was mixed with 20 g of a 2% aqueous chitosan solution also containing 1% aspirin. The gel was transferred into a plastic syringe and applied on to a standard lump for antimicrobial testing. Surprisingly, after 1 day and 5 days, no growth of either organism was observed. A zone of inhibition of about 3 to 4 mm was observed for E. coli. A zone of inhibition of about 4 to 6 mm was observed for S. aureus.

### Example 10

Antimicrobial Activity

20 g of an aqueous 35% PVP solution containing 0.5% Silver AlphaSan and 0.5% aspirin was mixed with 20 g of a 2% aqueous chitosan solution also containing 0.5% Silver AlphaSan and 0.5% aspirin. The gel was transferred into a plastic syringe and applied onto a standard lump for antimicrobial testing. After 1 day and 5 days, no growth of either organism was observed. A zone of inhibition of about 1 mm was observed for E. coli. A zone of inhibition of about 3 mm was observed for S. aureus.

### Example 11

Antimicrobial Activity

20 g of an aqueous 35% PVP solution containing 1% of a 48% commercially available Zinc Pyrithione solution (Zinc Omadine) was mixed with 20 g of a 2% aqueous chitosan solution also containing 1% of a 48% Zinc pyrithione solution. The gel was transferred into a plastic syringe and applied onto a standard lump for antimicrobial testing. After 1 day and 5 days, no growth of either organism was observed with a zone of inhibition of about 8 to 9 mm for E. coli, and 4 to 5 mm for S. aureus.

### Example 12

Antimicrobial Activity

20 g of an aqueous 35% PVP solution containing 0.05% Triclosan and 0.5% of a 40% commercially available 48% Zinc Pyrithione solution (Zinc Omadine) was mixed with 20 g of a 2% aqueous chitosan solution also containing 0.05% Triclosan and 0.5% of a 40% Zinc pyrithione solution. The gel was transferred into a plastic syringe and applied onto a standard lump for antimicrobial testing. After 1 day and 5 days, no growth of either organism was observed with a zone of inhibition of about 8 to 9 mm for E. coli, and 10 to 12 mm for S. aureus.

### Example 13

Antimicrobial Activity

20 g of an aqueous 35% PVP solution containing 0.05% Triclosan and 0.5% of antimicrobial silver AlphaSan was mixed with 20 g of a 2% aqueous chitosan solution also containing 0.05% Triclosan and 0.5% of antimicrobial silver AlphaSan. The gel was transferred into a plastic syringe and applied onto a standard lump for antimicrobial testing. After 1 day and 5 days, no growth of either organism was observed with a zone of inhibition of about 4 to 6 mm for E. coli, and about 1 mm for S. aureus.

### Example 14

Antimicrobial Activity

20 g of an aqueous 35% PVP solution containing 0.5% Zinc pyrithione and 0.5% of antimicrobial silver AlphaSan was mixed with 20 g of a 2% aqueous chitosan solution also containing 0.5% Zinc pyrithione and 0.5% of antimicrobial silver AlphaSan. The gel was transferred into a plastic syringe and applied onto a standard lump for antimicrobial testing. After 1 day and 5 days, no growth of either organism was observed with a zone of inhibition of about 3 to 4 mm for E. coli and about 6 mm for S. aureus.

### Example 15

Antimicrobial Activity

20 g of an aqueous 35% PVP solution containing no additional antimicrobial or drug was mixed with 20 g of a 2% aqueous chitosan solution also with no additional antimicrobial or drug. The gel was transferred into a plastic syringe and applied onto a standard lump for antimicrobial testing. Surprisingly, after 1 day and 5 days, no growth of either organism was observed. No growth was detected directly on the surface of the gel or on either side of the lump test sample. No zone of inhibition could be determined.

Thus, while there have been described what are presently believed to be the preferred embodiments of the present invention, other and further embodiments, modifications, and improvements will be known to those skilled in the art, and it is intended to include all such further embodiments, modifications, and improvements and come within the true scope of the claims as set forth below.

1. A hydrogel composition capable of preventing the intrusion of micro-organisms into body cavities or body openings of mammals comprising a poly(N-vinyl lactam), a polysaccharide and water.

2. The hydrogel composition of claim 1 wherein the upper boundary of the range of the ratio of the amount by weight of the poly(N-vinyl) lactam to the amount by weight of the polysaccharide is about 75:1; 50:1; 30:1; 20:1; 15:1; 13:1; 12:1; or 1:2.

3. The hydrogel composition of claim 1 wherein the lower boundary of the range of the ratio of the amount by weight of the poly(N-vinyl) lactam to the amount by weight of the polysaccharide is about 1:10; 1:5; 1:3, 1:1; 5:1; 12:1; 13:1; 15:1; 20: 1; 30:1; or 50:1.

4. The hydrogel composition of claim 1 comprising about 25 wt % to 90 wt % water; about 45 wt % to 75 wt % water; or about 55 wt % to 65 wt % water.

5. The hydrogel composition in claim 1 wherein the poly(N-vinyl lactam) is a homopolymer, a copolymer, a terpolymer of N-vinyl lactam, or mixtures thereof.

6. The hydrogel composition in claim 5 wherein the poly(N-vinyl lactam) is selected from the group consisting of N-vinylpyrrolidone, N-vinylbutyrolactam, N-vinylcaprolactam, and mixtures thereof.

7. The hydrogel composition in claim 5 wherein the poly(N-vinyl lactam) comprises a vinyl monomer copolymerized with the N-vinyl lactam.

8. The hydrogel composition in claim 7 wherein the vinyl monomer is selected from the group consisting of an acrylate, a hydroxyalkylacrylate, a methacrylate, an acrylic acid, a methacrylic acid, an acrylamide, and mixtures thereof.

9. The hydrogel composition in claim 5 wherein the homopolymer is polyvinylpyrrolidone (PVP).

10. The hydrogel composition in claim 5 wherein the copolymer is selected from the group consisting of a vinylpyrrolidone copolymer and an acrylamide copolymer.

11. The hydrogel composition in claim 5 wherein the terpolymer is selected from the group consisting of a vinylpyrrolidone terpolymer, a vinylcaprolactam terpolymer, and a dimethylaminoethyl methacrylate terpolymer.

12. The hydrogel composition of claim 1 wherein the polysaccharide is selected from the group consisting of chitin; deacetylated chitin; chitosan; chitosan salts; chitosan sorbate; chitosan propionate; chitosan lactate; chitosan salicylate; chitosan pyrrolidone carboxylate; chitosan itaconate; chitosan niacinate; chitosan formate; chitosan acetate; chitosan gallate; chitosan glutamate; chitosan maleate; chitosan aspartate; chitosan glycolate; quaternary amine substituted chitosan salts; N-carboxymethyl chitosan; O-carboxymethyl chitosan; N,—O-carboxymethyl chitosan; equivalent butyl chitosan derivatives; cellulosics, alkylcellulose; nitrocellulose; hydroxypropylcellulose; starch; starch derivatives; methyl gluceth derivatives; collagen, alginate; hialuronic acid; heparin; heparin derivatives; and combinations thereof.

13. The hydrogel composition in claim 2 further comprising a consistency modifying agent, a performance modifying agent, a cross-linker, or mixtures thereof.

14. The hydrogel composition in claim 13 wherein the consistency modifying and/or performance modifying agent is selected from the group consisting of polyvinyl alcohol; polyvinyl acetate; polyethylenoxide, poly(2-hydroxyethyl methacrylate); methyl vinyl ether-co-maleic anhydride; poly(ethylene-co-vinyl acetate); polyethylene glycol diacrylate; poly(N-isopropyl acrylamide; polyurethane; polyethylenimine; polypeptides; keratins; polyvinylpyrrolidone/polyethyleneimine; polyvinylpyrrolidone/polycarbamyl/-polyglycol ester;
polyvinylpyrrolidone/dimethylaminoethylmethacrylate/polycarbamyl/polyglycol ester; polyvinylpyrrolidone/dimethiconylacrylate/polycarbamyl/-polyglycol ester; lecithin; and copolymers, derivatives and combinations thereof.

15. The hydrogel composition in claim 13 wherein up to 5 wt %, 10 wt %, 20 wt %, 30 wt %, 40 wt %, 50 wt %, 60 wt %, 70 wt %, 80 wt %, or 90 wt % of the poly(N-vinyl lactam) is replaced with the consistency and/or performance modifying copolymers.

16. The hydrogel composition of claim 13 wherein the cross-linker is selected from the group consisting of glutaraldehyde, genipin, aziridine derivatives, carbodimid derivatives, colloidal silica, colloidal alumina, colloidal titanium dioxide, polyaminosilanes, epoxies, primary polyamines, dialdehydes, polyaldehydes from acrolein reaction products, paraformaldehyde, acrylamides, polyethylenimines, and combinations thereof.

17. The hydrogel composition of claim 1 further comprising a therapeutic performance enhancing agent.

18. The hydrogel composition of claim 17 wherein the therapeutic performance enhancing agent is selected from the group consisting of an antimicrobial, antibacterial, antifungal, anti-candidiasis agent, disinfecting agent, biocide, bactericide, preservative, virucide, spermicide, germicide, sterilant, sanitizing ingredient, deodorizer, antiseptic, sporicide, a pharmaceutical, a veterinary preparation, an antibiotic, an anti-inflammatory agent, a plant or seed extract, a plant extract derivative, an herbal preparation, a humectant, and combinations thereof.

19. The hydrogel composition of claim 18 wherein the therapeutic performance enhancing agent is selected from the group consisting of antimicrobial silver salts; silver zeolites; silver sulfadiazine; ethyl alcohol; isopropyl alcohol; benzyl alcohol; propionic acid; sorbic acid; salicylic acid; undecanoic acid; bleaches; iodine; iodophor; potassium iodide; dodecyl benzene sulfonic acid; peroxides; bronopol; terbinafine; miconacole; econacole; clotrimazole; tolnaphthate; triclosan; trichlocarban; quaternary ammonium compounds; benzalkonium halogenides; polyquats; polyquatemium derivatives; formaldehyde releasing compounds; hexetidin; chlorhexidine; chlorhexidine derivatives; zinc pyrithione; zinc oxide; zinc propionate; parabens; phenoxyethanol; octoxynol-9; nonoxynol-9; ricinoleic acid; phenol mercuric acetates; sulfur; lactic acid; essential oils of red thyme, allspice, cinnamon and savory; extracts of rosemary, echinechea, nettle, fennel, juniper, ginseng, borage, gelsemium, hamamelis, poke root, amica, aconite, apis, baptisia, thuja, aloe (barbadensis, vera, capensis), green tea, nasturtium, bryonia, eupatorium, and chamomile; acyclovir; idoxyumidine; ribavirin; vidarabine; rimantadine; aspirin; vitamin A and vitamin A derivatives; vitamin E and vitamin E derivatives; vitamin C and vitamin C derivatives; betacarotin; betamethasone; dexamethasone; cortinone; glycerin; and combinations thereof.

20. The hydrogel composition in claim 17 wherein the therapeutic performance enhancing agent comprises up to about 3 wt %, 7 wt %, 10 wt %, 15 wt %, or 20 wt % of the composition.

21. The hydrogel composition in claim 1 wherein 15 wt % to 75 wt %, 35 wt % to 65 wt %, or 45 wt % to 55 wt % of the water is replaced by ethyl alcohol or isopropyl alcohol.

22. The hydrogel composition of claim 1 further comprising a dye selected from the group consisting of a control dye, a food dye, a cosmetic dye, a FD&C dye or a D&C approved dye.

23. The hydrogel composition of claim 1 further comprising a radio-opaque additive selected from the group consisting of barium sulfate, iodine organics, iodine polymers, iodine contrast media, bismuth organics and tungsten particles.

24-32. (canceled)

33. A contraceptive hydrogel comprising a poly(N-vinyl lactam), a polysaccharide, water and a spermicide, wherein the ratio of the amount by weight of the poly(N-vinyl) lactam to the amount by weight of the polysaccharide is about 75:1 to 1:5; about 50:1 to 1:1; or about 30:1 to 5:1, and wherein the composition comprises about 25 wt % to 55 wt % water.
